(19) United States
(12) Patent Application Publication
(10) Pub. No.: US 2009/0238778 A1
(43) Pub. Date: Sep. 24, 2009

(54) TOOTH WHITENING COMPOSITIONS, DELIVERY SYSTEMS AND METHODS

(76) Inventors: Carolyn J. Mordas, Branchburg, NJ (US); Robert J. Gambogi, Hillsborough, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/051,016

(22) Filed: Mar. 19, 2008

(51) Int. Cl. A61K 8/22; A61K 8/49; A61Q 11/00

(52) U.S. Cl. 424/53; 424/49

(57) ABSTRACT

The present invention is directed to tooth whitening compositions that are capable of being activated by light energy, where the compositions include a safe and effective amount of riboflavin or a derivative of riboflavin, from about 0.1 to about 50 percent by weight of a bleaching agent and a water-soluble liquid phase; to systems for delivering the photoactivatable tooth whitening compositions to the surface of the teeth, and methods of whitening teeth that include the steps of exposing the photoactivatable whitening composition to light energy under conditions effective to photoactivate the tooth whitening composition and maintaining the composition in contact with the teeth under conditions effective to whiten teeth.

### FIELD OF THE INVENTION

This invention relates to novel tooth whitening compositions, delivery systems and methods that use light energy to provide improved tooth whitening.

### BACKGROUND OF THE INVENTION

White teeth have long been considered cosmetically desirable. Unfortunately, due to the presence of chromogenic (color-causing) substances in food, beverages, tobacco, and salivary fluid, in addition to internal sources such as blood, amalgam restoratives, and antibiotics such as tetracycline, teeth become almost invariably discolored in the absence of intervention. The tooth structures that are generally responsible for presenting a stained appearance are enamel, dentin, and the acquired pellicle. Tooth enamel is predominantly formed from inorganic material, mostly in the form of hydroxyapatite crystals, and further contains approximately 5% organic material primarily in the form of collagen. In contrast, dentin is composed of about 20% protein including collagen, the balance consisting of inorganic material, predominantly hydroxyapatite crystals, similar to that found in enamel. The acquired pellicle is a proteinaceous layer on the surface of tooth enamel which reforms rapidly after an intensive tooth cleaning.

Tooth stains may be either extrinsic or intrinsic, depending upon their location within the tooth structure. For example, extrinsic staining of the acquired pellicle arises as a result of compounds such as tannins and other polyphenolic compounds that become trapped in and/or tightly bound to the proteinaceous layer on the surface of the teeth. This type of staining can usually be removed by mechanical methods of tooth cleaning that remove all or part of the acquired pellicle together with the associated stain. In contrast, intrinsic staining occurs when chromogens or prechromogens penetrate the enamel and dentin and become tightly bound to the tooth structure. Intrinsic staining may also arise from systemic sources of chromogens or prechromogens, for instance, when excess fluoride intake during enamel development leads to the mottled yellow or brown spots typical of fluorosis staining. Intrinsic staining is not amenable to mechanical methods of tooth cleaning and generally requires the use of chemicals, such as hydrogen peroxide, that can penetrate into the tooth structure, in order to affect a change in the light absorptivity of the chromogen. Intrinsic tooth staining is generally more intractable and difficult to remove than extrinsic tooth staining.

Consequently, tooth-bleaching compositions generally fall into two categories: (1) gels, pastes, or liquids, including toothpastes, that are mechanically agitated at the stained tooth surface in order to affect tooth stain removal through abrasive erosion of stained acquired pellicle; and (2) gels, pastes, or liquids that accomplish the tooth-bleaching effect by a chemical process while in contact with the stained tooth surface for a specified period, after which the formulation is removed. In some cases, an auxiliary chemical process or additive, which may be oxidative or enzymatic, supplements the mechanical process.

Alternatively, there are oxidizing compositions (generally those with relatively high concentrations of oxidizers), which are applied directly to the tooth surface of a patient in a dental office setting under the supervision of a dentist or dental hygienist. The patient's soft tissues, e.g. the gingiva, lips, and other mucosal surfaces, must first be isolated from potential exposure to the active oxidizing agent by the use of a perforated rubber sheet (known as a rubber dam), through which only the teeth protrude. Alternatively, the soft tissue may be isolated from the oxidizers to be used in the whitening process by covering said soft tissue with a polymerizable composition that is shaped to conform to the gingival contours and subsequently cured by exposure to a high intensity light source. Once the soft tissue has been isolated and protected, the practitioner may apply the oxidizing agent directly onto the stained tooth surfaces for a specified period of time or until a sufficient change in tooth color has occurred. Results obtained through the use of in-office tooth whiteners are measured using the VITA® Shade Guide, VITA® Zahnfarbik, Bad Sackingen, Germany.

The range of tooth shades in the VITA® Shade Guide varies from very light (B1) to very dark (C4). A total of 16 tooth shades constitute the entire range of colors between these two endpoints on a scale of brightness. Patient satisfaction with a tooth whitening procedure increases with the number of tooth shade changes achieved.

Attempts have been made to activate peroxides with heat and/or light for the purpose of whitening teeth. U.S. Pat. No. 4,661,070 discloses a method of whitening stained teeth which includes the application of a concentrated solution of hydrogen peroxide within the pulp chamber or upon the surface of a discolored tooth, followed by exposing the discolored tooth to optical energy consisting of both ultraviolet and infrared light. The preferred wavelengths of light disclosed by this patent are from 320 to 420 nanometers and from 700 to 1200 nanometers, with light in the visible spectrum (wavelengths from 500 and 700 nanometers) being suppressed.

U.S. Pat. No. 5,032,178 discloses compositions and methods to improved tooth whitening efficacy which uses exposure to “optical energy”, preferably in the visible spectrum wavelength range of 400 to 700 nanometers. The compositions disclosed in this patent require the use of (1) an inert silica gelling agent, (2) a catalytic accelerator (either manganese sulfate monohydrate or ferrous sulfate), (3) an agent for providing thixoplasticity and thickening properties to the composition, such as cellulose ethers and methyl vinyl ethers, and (4) a means for indicating completion of the bleaching treatment of the teeth, comprising a redox color indicator for transforming from one color to another in response to the dissociation of hydrogen peroxide over a given time period. Compositions described therein are mixed homogeneously prior to use and all of the required components, including the catalyst, are dispersed evenly throughout the mixture. The compositions described are not highly transparent to light energy in the range of 400 to 700 nm, due to the presence of the high levels of inorganic silica particles.

A commercial product called Opalescence Xtra available for bleaching teeth in the controlled environment of a dental office has recently been introduced by Ultradent Products, Inc, South Jordan, Utah. The commercial product is supplied in a plastic syringe and is described in the accompanying literature as a light-activated tooth whitening gel, which contains approximately 35% hydrogen peroxide. A pH determination showed the product to have a neat pH at 25° C. of about 4.0. The product is thickened to a loose, gel-like consistency with a polymer. The product contains a bright orange pigment or dye (carotene), which presumably serves as the “photosensitizer”. The manufacturer also claims that the photosensitizer is able to absorb light energy and convert it into heat energy, thereby increasing the activity of the peroxide as a tooth bleaching agent.

There is thus a need for improved compositions, methods and devices for whitening teeth that overcome the limitations of the prior art described above. The compositions and methods of the present invention described herein satisfy these and other needs.

### SUMMARY OF THE INVENTION

The present invention is directed to tooth whitening compositions that are capable of being activated by light energy, i.e. photoactivatable, comprising a safe and effective amount of riboflavin or a derivative of riboflavin, from about 0.1 to about 50 percent by weight of a bleaching agent and a water-soluble liquid phase, to systems for delivering the tooth whitening compositions to the surface of teeth, and methods of whitening teeth including applying the photoactivatable tooth whitening composition to the surface of teeth, exposing the composition to light energy under conditions effective to photoactivate the tooth whitening composition and maintaining the photoactivated tooth whitening composition in contact with the teeth under conditions effective to whiten teeth.

### DETAILED DESCRIPTION OF THE INVENTION

By “oral care composition” or “oral composition” as used herein is meant a product which is not intentionally swallowed for purposes of systemic administration of therapeutic agents, but is retained in the oral cavity for a sufficient time to contact the dental surfaces for purposes of whitening teeth.

By “safe and effective amount” as used herein is meant an amount of a component, high enough to positively modify the condition to be treated or to effect the desired whitening result, but low enough to avoid serious side effects, within the scope of sound medical/dental judgment. The safe and effective amount of a component will vary with the particular condition being treated or the whitening effect sought, the age and physical condition of the patient being treated, the severity of the condition, the duration of treatment, the nature of concurrent therapy, the specific form employed, and the particular vehicle from which the component is applied.

By “exposure to conditions effective to photoactivate the tooth whitening composition” as used herein means exposure to light at a wavelength, light power and for a time effective to produce reactive moieties from the bleaching agent upon exposure of the photoactivatable tooth composition to such light.

By “maintaining the photoactivated tooth whitening composition in contact with the teeth under conditions effective to whiten the teeth” as used herein means that the photoactivated tooth whitening composition is in contact with the teeth for a sufficient period of time, whether upon a single application or multiple applications over a specified time interval, such that whitening of the teeth is achieved. For example, compositions may remain in contact with the teeth for greater than about 30 seconds, in another embodiment from about 2 minutes to about 12 hours (e.g overnight treatment), in another embodiment from about 3 minutes to about 120 minutes, in yet another embodiment from about 5 minutes to about 40 minutes, per application, and may be applied from about 1 to about 7 times per day. Additionally, the length of treatment to achieve the desired benefit, for example, tooth whitening, may last from about one day to about six months, in another embodiment from about one day to about 28 days, and in another embodiment from about 7 to about 28 days. The optimal duration and frequency of application will depend on the desired effect, the severity of any condition being treated, the health and age of the user and like considerations.

The compositions and methods of the present invention comprise a safe and effective amount of a riboflavin photoactivating agent. Riboflavin, also known as vitamin B2, has a chemical formula of C₁₇H₂₀N₄O₆. Derivatives of riboflavin may also be used in the present invention. One such compound is, but are not limited to riboflavin 5′-monophosphate. Others include, but are not limited to compounds that possess the chemical substructure shown in figure below:

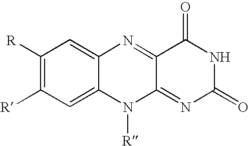

where R, R′, R″ can be any functional groups such that the electronic properties of the central three-ring structure are not altered significantly enough to prevent excitation, fluorescence, and activation of peroxide.

The riboflavin or riboflavin derivative photoactivating agent is generally used in compositions of the present invention at levels from about 0.0001% to about 10%, in another embodiment from about 0.0001% to about 0.5% by weight of the composition, and in yet another embodiment from about 0.001% to about 0.5% by weight of the composition.

A bleaching agent is also included in the compositions of the present invention. In one embodiment bleaching agents are selected from the group consisting of peroxides, metal chlorites, perborates, percarbonates, peroxyacids, persulfates, peroxyacids, organic peroxides or compounds that form the preceding compounds in situ, and combinations thereof. Suitable peroxide compounds include hydrogen peroxide, urea peroxide, calcium peroxide, carbamide peroxide, polyvinylpyrolidone-hydrogen peroxide complexes, copolymers of polyvinylpyrolidone complexed with hydrogen peroxide, other hydrogen peroxide complexes, and mixtures thereof. In one embodiment the bleaching agent is hydrogen peroxide. Suitable metal chlorites include calcium chlorite, barium chlorite, magnesium chlorite, lithium chlorite, sodium chlorite, potassium chlorite, and mixtures thereof Additional bleaching agents also include hypochlorite and chlorine dioxide. In one embodiment the bleaching agent is selected from sodium chlorite, peroxide, sodium percarbonate, oxones, and mixtures thereof The starting bleach agent can be aqueous or solid material.

It has been surprisingly found that combining riboflavin or derivatives of riboflavin with hydrogen peroxide enhances the whitening of teeth. Without being bound by theory, the present invention may increase the effective concentration of the bleaching agent on the surface of the teeth due to the ability of riboflavin to produce reactive moieties from hydrogen peroxide upon exposure to visible light. Therefore, increased speed of whitening and/or increased efficacy of the bleaching agent may be achieved, even though the same or lower total level of the bleaching agent is used. The present invention, therefore, at a given total concentration of bleaching agent, may require fewer applications to achieve the same degree of whitening, or may require a lower gel load to achieve the same degree of whitening.

It has also been surprisingly found that combining riboflavin or derivatives of riboflavin with hydrogen peroxide enhances the whitening of teeth at high pH values. Known compositions optimally perform at pH values of less than 7, typically between 4 and 6. The compositions described herein perform optimally in the pH range of about 6.5 to 9.0, indicating that the whitening activity of the combination is synergistically enhanced compared to either riboflavin or peroxide alone.

The level of the bleaching agent is dependent on the available oxygen that the molecule is capable of providing to bleach the stain. The bleaching agent is generally used in compositions of the present invention at levels from about 0.01% to about 50%, in another embodiment from about 0.1 to about 15% by weight of the composition.

The present invention comprises a safe and effective amount of a water-soluble liquid phase. Generally the level of the water-soluble liquid phase is from about 15% to about 99.9%, in another embodiment from about 20% to about 99%, in another embodiment from about 35% to about 99%, in another embodiment from about 45% to about 95%, by weight of the composition. In another embodiment the water-soluble liquid phase is from about 15% to less than about 90%, by weight of the composition.

The water-soluble liquid phase is generally selected from the group consisting of water, alcohols, polyalkylene glycols with molecular weights from about 200 to about 20,000, humectants, and mixtures thereof. Alcohols that can be used include ethanol and propanol. The water-soluble liquid phase can comprise water miscible components such as polyalkylene glycols, humectants, and mixtures thereof. Humectants generally include edible polyhydric alcohols such as glycerin, sorbitol, xylitol, butylene glycol, polyethylene glycol, and propylene glycol, and mixtures thereof. In one embodiment the water-soluble liquid phase is water. In one embodiment the composition comprises at least about 10% by weight water, in another embodiment at least about 20% by weight water.

The photoactivatable tooth whitening composition may be prepared prior to application to the delivery system. In such case, the riboflavin material and the bleaching agent are delivered as in a single composition. In other embodiments, the riboflavin material and the bleaching agent of the tooth whitening composition may be applied to the delivery system independently. In such a case, the photoactivatable composition is formed on the surface of the teeth after application of the delivery system to the teeth.

Optionally, the present invention may also comprise a safe and effective amount of a water-insoluble solid phase. Generally the water-insoluble solid phase comprises water-insoluble organic and/or inorganic (e.g. particulate) additives. The water-insoluble solid phase is generally water-insoluble, non-toxic to the user and chemically stable and compatible with the bleaching agent (e.g. does not cause substantial decomposition of the bleach agent) and the other ingredients present in the composition. Preferably, the insoluble phase has a refractive index matched with the liquid portion of the formulation for better light transmission.

Suitable materials for the water-insoluble solid phase include polyolefins (e.g., thermoplastic polymers, polymers derived from simple olefins, polyethylene, polypropylene, polyisoprenes, polybutene, and copolymers thereof) and polyesters, and mixtures thereof. Additional materials for the water-insoluble solid phase include water-insoluble celluloses (e.g. ethyl cellulose and cellulose acetate), silicas, talc, mica, magnesium carbonate, calcium carbonate, magnesium silicate, aluminum magnesium silicate, titanium dioxide, zinc oxide, nylon powder, methacrylate powder, polystyrene powder, silk powder, crystalline cellulose, titanated mica, calcium phosphate, calcium pyrophosphate, and mixtures thereof. In one embodiment the water-insoluble solid phase is selected from the group consisting of polyethylene, polypropylene, polyisoprenes, copolymers thereof, and mixtures thereof.

In one embodiment the water-insoluble solid phase is compatible with the bleaching agent such that it will not cause significant decomposition of the bleaching agent. In another embodiment a safe and effective amount of a stabilizer is added to the water-insoluble solid phase or to the composition of this invention to avoid decomposition of the bleaching agent. In another embodiment the level of optional stabilizer is from about 0.001% to about 15%, and in another embodiment from about 0.01% to about 10%, in another embodiment from about 0.5% to about 5%, in even another embodiment from about 1% to about 3%, by weight of the composition.

In one embodiment, suitable optional stabilizers, such as chelants or non-chelant stabilizers as known in the art, may be selected from the group consisting of tartaric acid and pharmaceutically acceptable salts thereof; citric acid and salts thereof such as alkali metal citrates; pyrophosphate ion source; polyphosphates (e.g., tripolyphosphate, hexametaphosphate); diphosphonates (e.g., EHDP; AHP); EDTA; and mixtures thereof; and in another embodiment may be selected from the group consisting of sodium citrate, potassium citrate, disodium tartrate, dipotassium tartrate, pyrophosphate ion source, sodium potassium tartrate, disodium hydrogen tartrate, potassium hydrogen tartrate, disodium dihydrogen pyrophosphate, tetrasodium pyrophosphate, tetrapotassium pyrophosphate, and mixtures thereof

When the water-insoluble solid phase is present, the ratio of the water-insoluble solid phase to the water-soluble liquid phase may be from about 1:10, in another embodiment from about 1:50, and in another embodiment from about 1:100.

The compositions and methods of the present invention optionally comprise a thickening agent. In one embodiment the thickening agent (or viscosity modifier) functions to increase retention of the composition on the teeth. The viscosity modifier may further function to inhibit settling and separation of components or control settling in a manner that facilitates re-dispersion and may control flow properties of the composition. A viscosity modifier is particularly useful to keep bleach agents or other oral care active agents that are in particulate form, suspended within the compositions of the present invention. The thickening agent herein can also serve as the adhesive means discussed herein below.

When present, the thickening agent (viscosity modifier) is present at a level of from about 0.01% to about 20%, in one embodiment from about 0.1% to about 10%, and in another embodiment from about 0.5% to about 5%, and in yet another embodiment from about 1% to about 3%, by weight of the composition.

Suitable viscosity modifiers herein include synthetic polymers such as carbomer polymers (e.g. crosslinked polyacrylic acid copolymer or homopolymer and copolymers of acrylic acid cross linked with a polyalkenyl polyether), natural polymer derivatives such as cellulose derivatives (e.g. methylcellulose, carboxymethylcellulose, hydroxyethylcellulose, hydroxypropylcellulose, hydroxy-propylmethylcellulose, etc.), natural and synthetic gums, karaya gum, guar gum, gelatin, algin, sodium alginate, tragacanth, chitosan, polyethylene oxide, acrylamide polymers, polyacrylic acid, polyvinyl alcohol, polyamines, polyquarternary compounds, ethylene oxide polymers, polyvinylpyrrolidone and its derivatives, cationic polyacrylamide polymers, and mixtures thereof.

In one embodiment the thickening agent is selected from carbomers. Carbomers are commercially available from B.F. Goodrich as the CARBOPOL series. In one embodiment the CARBOPOLs are CARBOPOL 934, 940, 941, 956, and mixtures thereof. Other examples of homopolymers which are useful include Ultrez 10, ETD 2050, and 974P polymers, which are available from B.F. Goodrich Company. Such polymers are homopolymers of unsaturated, polymerizable carboxylic monomers such as acrylic acid, methacrylic acid, maleic acid, itaconic acid, maleic anhydride, and the like.

In another embodiment the thickening agent can be an associative thickener or stabilizer, such as a hydrophobically modified alkali soluble acrylic emulsion or a hydrophobically modified nonionic polyol polymer, i.e., a hydrophobically modified urethane polymer, hydrophobically modified ethoxylated urethane polymer or combinations thereof. Associative thickeners may increase the retention or adhesion of compositions herein and/or integral carriers on the tooth surfaces, and may slow the erosion of the compositions once applied on the tooth surfaces.

Hydrophobically modified polyacrylic acid polymers have a large water-loving hydrophilic portion (the polyacrylic acid portion) and a smaller oil-loving hydrophobic portion (which can be derived from a long carbon chain acrylate ester). Representative higher alkyl acrylic esters are decycl acrylate, lauryl acrylate, stearyl acrylate, behenyl acrylate and melissyl acrylate, and the corresponding methacrylates. It should be understood that more than one carboxylic monomer and more than one acrylate ester or vinyl ester or ether or styrenic can be used in the monomer charge. The polymers can be dispersed in water and neutralized with base to thicken the aqueous composition, form a gel, or emulsify or suspend a deliverable. Useful polymers are sold as CARBOPOL 1342 and 1382, and CARBOPOL ETD 2020, and PEMULEN TR-1, TR-2, 1621, and 1622, all available from BF Goodrich. The carboxyl containing polymers are prepared from monomers containing at least one activated vinyl group and a carboxyl group, and would include copolymers of polymerizable carboxylic monomers with acrylate esters, acrylamides, alkylated acrylamides, olefins, vinyl esters, vinyl ethers, or styrenics. The carboxyl containing polymers have molecular weights greater than about 500 to as high as several billion, or more, usually greater than about 10,000 to 4,000,000, or more.

Also useful are interpolymers of hydrophobically modified monomers and steric stabilizing polymeric surface active agents having at least one hydrophilic moiety and at least one hydrophobic moiety or a linear block or random comb configuration or mixtures thereof Examples of steric stabilizers which can be used are Hypermerl, which is a poly(12-hydroxystearic acid) polymer, available from Imperial Chemical Industries Inc. and organosilicon compounds and emulsions thereof, sold under the trademark PECOSIL (Phoenix Chemical, Somerville, N.J.).

Other associative thickeners include those available from Rohm and Haas (such as ACRYSOL ICS-1 and ACULYN 22 and 28 thickeners, which are hydrophobically modified alkali-soluble acrylic polymer emulsions and ACULYN 44 and 46 thickener, which is a hydrophobically modified nonionic polyol). In one embodiment the associative thickener is CARBOPOL and/or PEMULEN polymers. The choice of the specific polymer to be employed will depend upon the desired rheology of the composition, and the identity of other compositional ingredients.

In one embodiment mixtures of hydrophobically modified carbomers and carbomers can be used.

In one embodiment the present invention relates to a delivery system comprising an integral carrier and the photoactivatable tooth whitening composition. In one embodiment the delivery system comprises: a first layer of a strip of material; a second layer comprising the photoactivatable tooth whitening compositions, whereby the bleaching agent is releasably associated with the composition and/or the strip of material. The present invention delivers whitening benefits to the oral cavity by directly applying the integral carrier to the teeth, and exposing the composition to light.

### I. First Layer

The first layer of the present invention comprises an integral carrier including a strip of material, dental tray, a sponge material, and mixtures thereof. In one embodiment the integral carrier is a strip of material.

The strip serves as a protective barrier for the bleach. It prevents leaching and/or erosion of the second layer by for example, the wearer's tongue, lips, and saliva. This allows the active in the second layer to act upon the hard surfaces of the oral cavity for an extended period of time, from several minutes to several hours.

The strip of material may comprise polymers, natural and synthetic woven materials, non-woven material, foil, paper, rubber and combinations thereof. The strip of material may be a single layer of material or a laminate of more than one layer. Regardless of the number of layers, the strip of material may be water-soluble, substantially water-insoluble or water erodible. The strip may also be water impermeable. Optical transparency is preferred for the materials in the strip of material.

In one embodiment the material is any type of polymer or combination of polymers that meet the required flexural rigidity and are compatible with oral care substances. Suitable polymers include, but are not limited to, polyethylene, polyethylvinylacetate, polyesters, poly(ethylvinyl alcohol) and combinations thereof. Examples of polyesters include MYLAR and fluoroplastics such as TEFLON, both manufactured by E. I. du Pont de Nemours and Company (Wilmington, Del.). In one embodiment the material is polyethylene. The strip of material is generally less than about 1 mm (millimeter) thick, in one embodiment less than about 0.05 mm thick, in yet another embodiment from about 0.001 to about 0.03 mm thick. A polyethylene strip of material is generally less than about 0.1 mm thick and in one embodiment from about 0.005 to about 0.02 mm thick.

The shape of the strip of material is any shape and size that covers the desired oral surface. In one embodiment the strip has rounded corners to avoid irritation of the soft tissue of the oral cavity. In one embodiment, the length of the strip material is from about 2 cm (centimeter) to about 12 cm, in another embodiment from about 4 cm to about 9 cm. The width of the strip material will also depend on the oral surface area to be covered. The width of the strip is generally from about 0.5 cm to about 4 cm, in one embodiment from about 1 cm to about 2 cm. In yet another embodiment, the strip may be worn as a patch on one or several teeth to treat a localized condition.

The strip material may contain shallow pockets. When the composition is coated on a strip of material, bleach agent and/or oral care actives fill shallow pockets to provide reservoirs of additional bleach agent and/or oral care actives. Additionally the shallow pockets help to provide texture to the delivery system. In one embodiment the strip material will have an array of shallow pockets. Generally the shallow pockets are approximately 0.4 mm across and about 0.1 mm deep. When shallow pockets are included in the strip of material and the compositions herein are applied to it in various thicknesses, the overall thickness of the delivery system is less than about 1 mm. In one embodiment the overall thickness is less than about 0.5 mm.

In one embodiment the delivery systems herein comprise an adhesion means and are capable of adhesion to oral surfaces especially the teeth. This adhesion means may be provided by the present compositions herein or the adhesion means is provided independently of the compositions herein (for example the adhesion means is a separate phase from the compositions herein where the compositions may or may not also have an adhesive means). In one embodiment the strip of material is held in place on the oral surface by adhesive attachment provided by the present compositions. The viscosity and general tackiness of the present compositions to dry surfaces cause the strip to be adhesively attached to the oral surface without substantial slippage from the frictional forces created by the lips, teeth, tongue, and other oral surfaces rubbing against the strip of material while talking drinking, etc. However, this adhesion to the oral surface is low enough to allow the strip of material to be easily removed by the wearer. The delivery system is easily removable from the oral surfaces without the use of an instrument, a chemical solvent or agent or excess friction.

In another embodiment the strip of material is held in place on the oral surface by adhesive means and attachment provided by the integral carrier itself. In one embodiment the strip of material can extend, attach, and adhere to the oral soft tissue. Alternatively, an adhesive can be applied to that portion of the strip of material that will attach the delivery systems to the oral soft tissue

Examples of adhesion means being provided independent of the compositions herein include the following.

In one embodiment, the composition and an adhesive material may be deposited in separate discrete locations in relation to the strip surface. In one embodiment the composition and adhesive may be deposited on the surface of the strip in respective spatially separated places on the surface. For example the adhesive may be deposited in places on the strip surface that enable part of the strip to stick to an oral surface adjacent to a tooth surface, e.g. a gum surface, so that another part of the strip on which the composition is deposited or into which it is impregnated may contact the tooth surface.

Alternatively the adhesive and composition may be spatially separated but both in locations that enable the adhesive and composition to contact the same type of tissue, e.g. tooth or gum surface, respective discrete spots or patches on the surface, that are relatively small. For example parallel lines of the adhesive and composition, one or more patches of composition bordered partly or completely surrounded by a border of the adhesive, a single large patch covering substantially the entire surface of the strip and bordered partly or completely by a line of the adhesive. Adhesive may be deposited on one or more patch bordered partly or completely surrounded by a border of the composition.

In another embodiment the composition and/or adhesive may be encapsulated. Encapsulation may for example be in micro-capsules, or macro-capsules. Methods of micro-encapsulation are known, for example in which a droplet of a substance in a liquid phase is enclosed within a layer of an encapsulation material, and then separated from the liquid. Such capsules may be deposited on or adjacent the surface of the integral carrier, and may for example be burst physically or chemically, e.g. by pressure e.g. as the strip is applied to the tooth surface or by subsequent bit action, by breaching of the capsule wall under the action of the temperature, moisture, pH, chemicals or enzymes in the mouth environment etc. For example respective capsules of composition and adhesive may be attached to the surface of the strip, e.g. by means of a second adhesive or by embedding the capsules in the strip of material. For example a thin layer of the adhesive may be deposited on the surface of the strip, and capsules of the composition may be embedded at least partly if not completely within this adhesive layer, or may sit upon the surface of this adhesive layer.

In another embodiment the adhesive may be provided in granules, e.g. pellets or micropellets, which may release their content under the influence of the mouth environment, for example moisture, chemicals or enzymes in the mouth, and may be coated to achieve this release. Methods of granulation and palletizing are known, as are coating polymers such as the know Eudragit® polymers which dissolve at specified pH. Such adhesive granules may be deposited on or adjacent the surface of the strip. For example capsules and/or granules of adhesive may be located substantially uniformly over the strip surface, or alternatively respective capsules and/or granules of adhesive may be situated at separate respective locations on the surface of the strip.

In another embodiment a layer of the composition may be deposited relatively proximal to, e.g. adjacent to and in contact with the surface, and a layer of the adhesive may be deposited relatively distal from the surface e.g. adjacent to and in contact with the underlying layer of composition. In such a construction the adhesive may stick the strip to the tooth surface, and the composition may pass through the adhesive layer, for example as the adhesive layer becomes permeable under the influence of the mouth environment. The adhesive layer may in such a construction have one or more holes passing through the layer to facilitate the passage of the composition through the adhesive layer. Alternatively for example a layer of the adhesive may be deposited relatively proximal to, e.g. adjacent to and in contact with the surface, and a layer of the composition may be deposited relatively distal from the surface e.g. adjacent to and in contact with an underlying layer of adhesive. In such a construction the layer of composition may need one or more holes passing through the layer to facilitate the passage of the adhesive through the composition. In the above constructions the passage of material from the underlying layer may be facilitated by pressure as the strip is applied to the tooth surface.

Mechanical adhesive means may also be used to provide an adhesive function, used either alone or in combination with any other adhesive device disclosed herein. In another embodiment mechanical adhesion between the strip and tooth or other oral surface is provided by the strip comprising a plastically deformable material, which can be plastically deformed by the user to conform the strip to the contours of the tooth or other oral surface, and so adhere thereto by mechanical gripping. Such gripping may be enhanced by e.g. surface effects between the strip and the surface such as formation of a partial vacuum or surface tension effects. For example the strip may have anchors on its surface, positioned at approximately the spacings of gaps between teeth, and these anchors may fit into the gaps between the teeth. For example the surface of the strip which is to contact the tooth surface may be provided with micro-suckers, that is a plurality of small cavities in the surface of the strip which can be pressed onto the tooth surface to drive air out therefrom, and thereby create a partial vacuum, so that the strip is thereafter held on the tooth surface by air pressure. Such anchors or micro-suckers may be located on the surface of a strip which is to contact the tooth surface. Such a strip may for example be stretchable, so that it can be adjusted to the spacings of gaps between an individual user's teeth. Another form of “mechanical” adhesion may be provided by a strip which shrinks in contact with the tooth surface, so it can physically grip the surface of the tooth.

When the adhesive means is provided by an adhesive, the adhesive may be any adhesive which may be used to stick materials to the tooth surface or to a surface of the oral cavity surfaces. Suitable adhesives include skin, gum and muco adhesives, and should be able to withstand the moisture, chemicals and enzymes of the oral environment for long enough for the oral care actives and/or bleach to take effect, but may be soluble and/or biodegradable thereafter. Suitable adhesives may for example comprise water-soluble polymers, hydrophobic and/or non-water-soluble polymers, pressure and moisture sensitive adhesives, e.g. dry adhesives which become tacky upon contact with the mouth environment, e.g. under the influence of moisture, chemicals or enzymes etc. in the mouth. Suitable adhesives include natural gums, synthetic resins, natural or synthetic rubbers, those gums and polymers listed above under “Thickening Agents”, and various other tacky substances of the kind used in known adhesive tapes, such as those known from U.S. Pat. No. 2,835,628.

### II. Second Layer

In one embodiment the second layer comprises a safe and effective amount of the present composition described herein.

### Optional Release Liner

The release liner may be formed from any material which exhibits less affinity for the second layer composition than the second layer composition exhibits for itself and for the first layer strip of material. The release liner may comprise a rigid sheet of material such as polyethylene, paper, polyester, or other material, which is then coated with a nonstick type material. The release liner may be cut to substantially the same size and shape as the strip of material or the release liner may be cut larger than the strip of material to provide a readily accessible means for separating the material from the strip. The release liner may be formed from a brittle material that cracks when the strip is flexed or from multiple pieces of material or a scored piece of material. Alternatively, the release liner may be in two overlapping pieces such as a typical adhesive bandage design.

### Combination of Soft/Rigid Dental Trays or Sponge Material (Foams) and Composition

The delivery systems may be used in combination with a dental tray. Dental trays are well known in the whitening art. The general process for preparing dental trays is known in the art. For example, an alginate impression which registers all teeth surfaces plus gingival margin is made and a stone cast is promptly made of the impression. If reservoirs are desired they are prepared by building a layer of rigid material on the stone cast on specific teeth surfaces to be treated. A custom dental tray is then vacuum-formed from the modified cast using conventional techniques. Once formed, the tray is preferably trimmed barely shy of the gingival margin on both buccal and lingual surfaces. Preferably enough tray material should be left to assure that all of the tooth will be covered to within about ¼ to about ⅓ mm of the gingival border upon finishing and beveling the tray periphery. In one embodiment one can scallop up and around interdental papilla so that the finished tray does not cover them. All tray edges are preferably smoothed so that the lip and tongue will not feel an edge prominence. The resulting tray, in one embodiment, provides a perfect fit of the patient's teeth optionally with reservoirs or spaces located where the rigid material was placed on the stone cast. Dental trays may comprise of soft transparent vinyl material having a preformed thickness from about 0.04 inch to about 0.06 inch. Soft material is more comfortable for the patient to wear. Harder material (or thicker plastic) may also be used to construct the tray.

Dentists have traditionally utilized three types of dental appliances for bleaching teeth. The first type is a rigid appliance which is fitted precisely to the patient's dental arches. A second type of rigid custom dental appliance is an “oversized” rigid custom dental appliance. The fabrication of rigid, custom dental appliances entails fabricating stone models of the patient's dental arch impressions, and heating and vacuum-forming a thermoplastic sheet to correspond to the stone models of a patient's dental arches. Thermoplastic films are sold in rigid or semi rigid sheets, and are available in various sizes and thickness. The dental laboratory fabrication technique for the oversized rigid dental appliance involves augmenting the facial surfaces of the teeth on the stone models with materials such as die spacer or light cured acrylics. Next, thermoplastic sheeting is heated and subsequently vacuum formed around the augmented stone models of the dental arch. The net effect of this method results in an “oversized” rigid custom dental appliance.

A third type of rigid custom dental appliance, used with less frequency, is a rigid bi-laminated custom dental appliance fabricated from laminations of materials, ranging from soft porous foams to rigid, non-porous films. The non-porous, rigid thermoplastic shells of these bi-laminated dental appliances encase and support an internal layer of soft porous foam.

A fourth type of dental tray replaces rigid custom dental appliances with disposable U-shaped soft foam trays, which may be individually packaged, and which may be saturated with a pre-measured quantity of the composition of the present invention. The soft foam material is generally an open celled plastic material. Such a device is commercially available from Cadco Dental Products in Oxnard, Calif. under the tradename VITALWHITE. In one embodiment these soft foam trays comprise a backing material (e.g. a closed cell plastic backing material) to minimize the elution of the bleaching agent from the device, into the oral cavity to minimize ingestion by the patient and/or irritation of the oral cavity tissues. In another embodiment the soft foam tray is encased by a nonporous flexible polymer. In another embodiment the open cell foam is attached to the frontal inner wall of the dental appliance and/or the open cell foam is attached to the rear inner wall of the dental appliance.

Those of ordinary skill in the art will readily recognize and appreciate, that the present compositions must be thick enough not to simply run out between the open cell structure of the foam and must be thin enough to slowly pass through the open cell foam over time. In other words, the open cell foam material has an internal structural spacing sized relative to the viscosity of the compositions to absorb and allow the composition to pass therethrough.

An example of a closed cell material is a closed-celled polyolefin foam sold by the Voltek division of Sekisui America Corporation of Lawrence, Mass. under the tradename VOLORA which is from 1/32 to ⅛-inch in thickness. A closed cell material may also comprise of a flexible polymeric material.

An example of an opened cell material is an open celled polyethylene foam sold by the Sentinel Foam Products division of Packaging Industries Group, Inc. of Hyannis, Mass. under the tradename OPCELL which is from 1/16 to ⅜-inch in thickness. The above dental appliances may be designed to be disposable or reuseable.

### Liquid Compositions

In instances where the tooth whitening composition is a liquid composition, the composition generally includes between about 1% and about 50% by weight of carrier compound, between about 0.1% and 35% by weight of tooth whitening agent, and between about 40% and about 99.9% by weight of a solvent.

In some embodiments, the liquid composition includes a multi-component mixture where each component is maintained separately prior to application of the whitening composition on the tooth. For example, in some embodiments it is desirable to prevent contact of the tooth bleaching agent with another ingredient in the composition, for example a colorant, flavorant or anticarries agent, in order to prevent the peroxide from reducing the activity of the second ingredient. Accordingly, in some embodiments, the tooth whitening composition includes two or more component mixtures where in a two component mixture the first component is a liquid mixture including the tooth bleaching agent and the second, separate component includes one or more of a colorant, flavorant, or anticarries agent. The two components are combined upon application to the tooth.

In some embodiments, the liquid composition is left on the tooth of the user until it dissolves in the user's mouth, thus requiring no active removal step from the user. In other embodiments, after the composition has been on the tooth for a desired amount of time, the composition is removed using a brush or a rinsing agent. The composition may remain in contact with a tooth, for example, for at least about 1 minute, for example at least about 5 minutes, at least about 10 minutes, at least about 30 minutes, at least about 1 hour, at least about 2 hours, at least about 4 hours, at least about 8 hours, or overnight.

The liquid composition can be applied, for example, directly onto the tooth without a protective shield. Alternatively, the liquid composition can be applied on the tooth using a dental tray to keep the tooth whitening composition in contact with the tooth for a desired amount of time. Alternatively, the liquid composition can be applied using a light emitting toothbrush, tongue scraper or similar dental devices.

The liquid composition can be made, for example, by combining the ingredients in the composition to form a stable liquid. In general, the liquid composition is prepared by first combining a solvent such as ethanol with a tooth bleaching agent such as hydrogen peroxide. A cellulose compound is then added and mixed until it is dissolved. The pH is adjusted, for example with sodium hydroxide. Flavors, dyes or other ingredients are added as appropriate and q.s. with solvent upon completion.

### Film Compositions

In some embodiments, the tooth whitening composition is in the form of a film. The film may include, for example, a single, unitary layer, or alternatively can include multiple layers, for example a dual layer film or a film containing three or more layers. The film may include, for example, a polymer such as polyvinylpyrolidone, a tooth bleaching agent, and a plasticizer.

The film may be comprised of between about 1% and about 85% by weight of the polymer. Examples of preferred polymers include polyvinylpyrolidone, hydroxypropylmethylcellulose esters, for example phthalate, succinate, or acetate esters thereof. Polymers that are traditionally used as enteric coatings that are stable in the presence of an oxidative moiety would also be good examples.

The bleaching agent may be present in the film at, for example, between about 0.1% and about 35% by weight of the film.

The plasticizer may be present in the film at, for example, between about 0.1% and about 30% by weight of the film. Examples of preferred plasticizers include polyols such as polyethylene glycol such as PEG-400.

The film may be made, for example, by making a solution of the film forming polymer, a plasticizer, and a tooth bleaching agent dissolved in a casting solvent, for example ethanol or ethanol, water mixtures. The solution is then poured into a case, for example a processing liner, where the casting solvent is substantially removed during the forming of the film.

In some embodiments, where the film is a unitary film, the components are evenly distributed throughout the tooth whitening strip. In other embodiments, for example where the strip is a dual layer strip the components can be separated into individual layers of the film. For example a dual layer film can include a first, tooth contacting layer that includes an adhesive and a tooth bleaching agent. The second layer can include a cellulose compound that acts a barrier to keep the tooth bleaching agent in contact with the tooth and prevent the tooth bleaching agent from becoming washed away from the tooth in the saliva of the user before the tooth bleaching agent has acted upon the tooth. In still other embodiments, the film includes three or more layers, each layer can include one or more of a tooth bleaching agent, oral active ingredient, adhesive, dye, cellulose compound, etc. Optional aesthetic layers are also considered (e.g. external flavoring layer).

The backing layer may include a thin, flexible layer of permanently deformable material. As used herein “permanently deformable” means that the backing layer retains any shape into which it is formed by application of slight pressure e.g., less than about 250,000 Pascals per square centimeter. Thus, the film can readily conform to the surface of the teeth and adjoining soft tissue across the dental arch in the user's mouth without tearing, cracking or breaking. The backing layer material generally has visco-elastic properties which allow the backing layer to creep as well as bend when pressure is applied to the device. The backing layer can include a non-polymeric material such as a wax (e.g., microcrystalline or paraffin waxes), a tackifier (e.g., a natural or synthetic resin, such as a hydrocarbon resin), a hydrocarbon resin, or mixtures thereof.

In some embodiments, the backing layer is flavored. For example, the backing layer can include a flavorant forumulated therein. The flavorant can be incorporated, for example, into a compound, which releases the flavorant into the mouth of the user. The formulated flavorant can be cast as a solution onto a wax in the backing layer or laminated onto the backing.

In general, the flavorant formulation includes from about 1% to about 50% by weight compound, from about 1% to about 30% by weight of flavorant, such as a flavor oil, and from about 20% to about 90% solvent, such as ethanol (e.g., 190 proof ethanol). The formulated flavor can be uniformly distributed on the backing layer, and/or can be applied in a patterned manner such as stripes or polka-dots. The intensity and amount of flavor can be adjusted, for example, by controlling the thickness of the cast film or modification of the dissolution properties or diffusive properties of the flavorant matrix.

In some embodiments, the backing layer can include an oral care active ingredient such as an anticaries agent or an antimicrobial agent in addition to or in place of the flavorant. Agents that help to reduce oral malodor are considered for some embodiments.

The anchor layer generally includes a thin, flexible layer of material such as open-cell foam or a non-woven material (e.g., polyester or polypropylene) which is located immediately adjacent to the backing layer. The opposing faces of the backing and anchor layer material are in contact with one another essentially along their entire surfaces, and the material comprising the backing layer penetrates slightly into spaces between the cells of the opposing side of the foam forming the anchor layer. Generally, the face of the backing layer contacting the anchor layer is softened by heating prior to laminating the backing layer to the anchor layer.

The oral care layer generally includes at least one oral care agent and at least one hydrophilic polymer, and is located immediately adjacent to the side of the anchor layer which is not attached to the backing layer. The opposing faces of the oral care and anchor layers are in contact with one another and form an adhesive bond between those layers. The oral care layer is disposed on the anchor layer and is minimally invested in the foam. As used herein, “minimally invested” means that the oral care layer fills only the surface depressions in the anchor layer foam, but does not appreciably penetrate below the surface of the anchor layer. The oral care layer is generally co-extensive with the anchor layer.

The rate at which the bleaching agent is solubilized (in the case of a solid bleaching agent) and subsequently released to a tooth surface can be controlled by properties such as, but not limited to, the film thickness, compound properties such as structure and molecular weight and type, properties of the bleaching agent and the concentration of the bleaching agent.

The thicknesses of the film layer may affect the residence time of the composition. The residence time of a layer including the cellulose compound may, for example, be determined by a combination of the composition of the compound and the thickness of the film. However, inclusion of a water-soluble, compound precast film as one of the layers of the device may also increase the residence time, since the overall thickness of the device is increased.

The total thickness of the film may, for example, affect the mouth feel of the product, which can relate to user acceptance and compliance. A very thick device can become more noticeable with respect to “mouth feel”, and may cause the user to discontinue its use prematurely, thus compromising efficacy.

Additionally, the thickness of the film may also affect its ability to conform and adhere to teeth in an efficient manner. Typically, thin films are preferred since they can be easily applied and bent around the teeth, minimizing the amount of unattached area in the form of edges and comers that could cause the film to be accidentally pulled off the teeth. However, if the film is too thin, it may lose tensile strength and rip during its application. In general, a preferred thickness for the film is between about the overall thickness of the device is between about 20 micrometers and about 1500 micrometers, and more preferably between about 50 micrometers and about 1000 micrometers.

The film can be applied to the tooth, for example, by pressing the film onto the tooth of the user. In some embodiments, the film will dissolve in the mouth of the user, and thus will not require a separate removal step, for example using a brush or a rinse. In other embodiments, the film will not completely dissolve in the mouth of a user and will require removal by the user, for example, by simply pulling of the remaining strip or using a brush or rinse.

The film may remain in contact with the tooth, for example, for at least about 2 minutes, for example at least about 5 minutes, at least about 10 minutes, at least about 30 minutes, at least about 1 hour, at least about 2 hours, at least about 4 hours, or overnight.

### Exposure to Light

The oral care composition of the present invention is activated when exposed to light. Any source of actinic light capable of emitting light of wavelength 250-700 nm, preferably 400-500 nm, can be used. Optimal wavelengths for photoactivation may vary for riboflavin derivatives. In one embodiment, a blue LED emitting 425-475 nm can be used. In another embodiment, an argon ion laser emitting blue light can be used. The light power should be from 0.1 mW/cm² to 100 mW/cm², preferably from 1.0 mW/cm² to 20.0 mW/cm². The duration of exposure of the photoactivatable whitening composition to light may be from about 30 seconds to about 60 minutes, or from about 2 minutes to about 45 minutes, or from about 5 minutes to about 30 minutes. The means of exposing the composition to light include, but is not limited to, the use of collimated lenses to apply a non-disperse beam, applying dispersed light, and applying coherent laser light to the tooth surface.

In other embodiments, other actives can be included in the composition. These include, but are not limited to: anti-calculus agents; fluoride ion sources; stannous ion source; anti-microbial agents; anti-plaque agents; anti-inflammatory agents; anti-adhesion agents; nutrients; antioxidants; anti-viral agents; anti-fungal agents; analgesic and anesthetic agents; H-2 antagonists; components which impart a clean feel to the teeth; fragrances and sensates; flavorants; sweeteners; and mixtures thereof.

### EXAMPLES

Whitening compositions for teeth and methods of use illustrated in following examples illustrate specific embodiments of the compositions of the present invention, but are not intended to be limiting thereof. Other modifications can be undertaken by the skilled artisan without departing from the spirit and scope of this invention.

### Example 1

### Enhanced Formation of Reactive Species with Riboflavin

The generation of hydroxyl radicals was determined through the use of sodium salicylate as a radical trap. (J. Chrom. A. 1998, 796, 283-288). The following modified HPLC method for salicylate was used.

HPLC mobile phase was prepared by adding 225 mg of tetramethylammonium hydroxide pentahydrate to 150 milliliter of acetonitrile, 150 milliliter of methanol, 700 milliliter of purified water, and 2 milliliter of glacial acetic acid, followed by mixing. The reversed-phase column used was either a Thermo Electron Corporation Betasil C18, P/N 70103-154630 or Phenomenex Gemini C18, P/N 00F-4439-Y0, and the column compartment temperature was maintained at 4020 C. for the duration of the run. The injection volume was set to 10 microliter. The retention time of salicylate using the above mobile phase and column was approximately 6-7 minutes with a 0.8 milliliter/min flow rate.

The following stock solution was prepared:
1A) Salicylate stock solution: 0.10 grams of sodium salicylate were added to a 100 milliliter volumetric flask and diluted to volume with purified water.

The following four formulations were then prepared using the stock solution:
1B) Control solution: 4.0 milliliter of solution 1A was transferred to a 100 milliliter low-actinic volumetric flask and diluted to volume with 0.05 M pH 8.0 phosphate buffer.
1C) 0.6% hydrogen peroxide solution: 4.0 milliliter of solution 1A and 20 milliliter of 3.0% hydrogen peroxide were added to a low-actinic 100 milliliter volumetric flask and then diluted to volume with 0.05 M pH 8.0 phosphate buffer.
1D) 0.27 mM Riboflavin: 4.0 milliliter of solution 1A and 10.0 mg of riboflavin powder was added to a 100 milliliter low-actinic volumetric flask and diluted to mark with 0.05 M pH 8.0 phosphate buffer.
1E) 0.27 mM Riboflavin plus 0.6% H2O2: 4.0 milliliter of solution 1A, 20.0 milliliter of 3.0% hydrogen peroxide and 10.0 mg of riboflavin powder was added to a 100 milliliter low-actinic volumetric flask and diluted to mark with 0.05 M pH 8.0 phosphate buffer.

Formulations 1B to 1E were irradiated with visible light (450 nm +/− 25 nm, 2 mW) for thirty minutes and the hydroxylation of salicylate with hydroxide radical was monitored through a loss of salicylate chromatographically using the conditions described above. The results are shown on Table 1.

TABLE 1
Hydroxylation of salicylate.

| | Milligram of oxidized salicylate formed |
|---|---|
| Solution | relative to control (1B) |
| 1C - Peroxide only | 0.01 |
| 1D - Riboflavin only | 1.83 |
| 1E - Peroxide and Riboflavin | 3.52 |

The data shows that more reactive species are generated with irradiated (450 nm) riboflavin formulations (ID) versus the peroxide control formulations (1C). Of note is that irradiated (450 nm) riboflavin plus peroxide formulations (1E) synergistically produced more radical species as monitored with this assay.

### Example 2

### Enhanced Formation of Reactive Species with Riboflavin 5′-monophosphate

The following stock solution was prepared:
2A) Salicylate stock solution: 0.10 grams of sodium salicylate were added to a 100 milliliter volumetric flask and diluted to volume with purified water.

The following four formulations were then prepared using the stock solution:
2B) Control solution: 4.0 milliliter of solution 2A was transferred to a 100 milliliter low-actinic volumetric flask and diluted to volume with 0.05 M pH 8.0 phosphate buffer.
2C) 0.6% hydrogen peroxide solution: 4.0 milliliter of solution 2A and 20 milliliter of 3.0% hydrogen peroxide were added to a low-actinic 100 milliliter volumetric flask and then diluted to volume with 0.05 M pH 8.0 phosphate buffer.
2D) 0.63 mM Riboflavin 5′-monophosphate: 4.0 milliliter of solution 2A and 30.0 mg of riboflavin phosphate powder was added to a 100 milliliter low-actinic volumetric flask and diluted to mark with 0.05 M pH 8.0 phosphate buffer.
2E) 0.63 mM Riboflavin 5′-monophosphate plus 0.6% H2O2: 4.0 milliliter of solution 2A, 20.0 mL of 3.0% hydrogen peroxide and 30.0 milligram of riboflavin phosphate powder was added to a 100 milliliter low-actinic volumetric flask and diluted to mark with 0.05 M pH 8.0 phosphate buffer.

Formulations 2A to 2E were irradiated with visible light (450 nm +/−25 nm, 2 mW) for thirty minutes and the hydroxylation of salicylate with hydroxide radical was monitored through a loss of salicylate chromatographically using the conditions described above. The results are shown on Table 2.

TABLE 2
Hydroxylation of salicylate.

| | Milligram of oxidized |
|---|---|
| | salicylate formed |
| Solution | relative to control (2B) |
| 2C - Peroxide only | 0.02 |
| 2D - Riboflavin 5′-monophosphate only | 1.91 |
| 2E - Peroxide and Riboflavin 5′- | 2.34 |
| monophosphate | |

The data shows that more reactive species are generated with irradiated (450 nm) riboflavin 5′-monophosphate formulations (2D) versus the peroxide control formulations (2C). Of note is that the irradiated (450 nm) riboflavin 5′-monophosphate plus peroxide formulations (2E) synergistically produced more radical species as monitored with this assay.

### Example 3

### Impact of Enhanced Generation of Reactive Species on Tooth Whitening

Prototype formulations containing riboflavin a riboflavin derivative and peroxide were prepared and evaluated in-vitro in a bovine tooth whitening model:

Formulation 3A (Control): 4.0 grams of CARBOPOL 974P was slowly added to 380 grams of 3.0% H2O2 with mixing and gentle heating. After all the carbopol was incorporated, the mixture was cooled to room temperature and the pH of the formulation was adjusted to 6.0 with 2 N NaOH.

Formulation 3B (Riboflavin): 4.0 grams of CARBOPOL 974P was slowly added to 380 grams of 3.0% H2O2 with mixing and gentle heating. After all the carbopol was incorporated, the mixture was cooled to room temperature and 39 milligram of riboflavin was added. The pH of the formulation was adjusted to 6.0 with 2 N NaOH.

Formulation 3C (Riboflavin 5′-monophosphate): 4.0 grams of CARBOPOL 974P was slowly added to 380 grams of 3.0% H2O2 with mixing and gentle heating. After all the carbopol was incorporated, the mixture was cooled to room temperature and 48 milligram of riboflavin was added. The pH of the formulation was adjusted to 6.0 with 2 N NaOH.

The prototype formulations listed above were applied to bovine enamel and exposed either to ambient light or to 450 nm +/−25 nm irradiation at 1.5 mW/cm² for 20 minutes per treatment. The color of the stain on the bovine teeth was measured by taking diffuse reflectance absorbance readings with a SpectroShade Micro spectrophotometer (MHT, Italy). Absorbance measurement over the entire visible color spectrum were obtained using the CIELAB color scale (Commission International de L'Eclairage, 1978 and 1986). This scale quantifies color according to 3 parameters, L* (lightness-darkness); a* (red-green); and b* (yellow-blue) with increasing L* and decreasing b* being more aesthetically pleasing and desirable. Table 3 shows the change in the L* values (ΔL) with each treatment for the three formulations with exposure to ambient light or 450 nm irradiation at 1.5 mW/cm².

TABLE 3
Bovine teeth bleaching.

| | | □L-1 | □L-2 | □L-3 |
|---|---|---|---|---|
| | Light | treatment | treatments | treatments |
| 3A - Peroxide only | Ambient | 1.83 | 2.24 | 2.79 |
| 3B - Peroxide and Riboflavin | Ambient | 2.27 | 2.67 | 3.52 |
| 3C - Peroxide and Riboflavin | Ambient | 1.84 | 2.08 | 2.47 |
| 5′-monophosphate | | | | |
| 3A - Peroxide only | 450 nm @ 1.5 mW/cm2 | 2.73 | 3.88 | 4.57 |
| 3B - Peroxide and Riboflavin | 450 nm @ 1.5 mW/cm2 | 3.55 | 5.13 | 5.93 |
| 3C - Peroxide and Riboflavin | 450 nm @ 1.5 mW/cm2 | 2.89 | 3.47 | 3.97 |
| 5′-monophosphate | | | | |

The table shows that all three formulations lighten teeth. There was an enhancement in tooth whitening in the presence of Riboflavin, as well as enhanced whitening when 450 nm irradiation at 1.5 mW/cm2 was used. While riboflavin 5′-monophosphate at first appears to be less effective in whitening than peroxide only or peroxide in combination with riboflavin, it should be noted that riboflavin 5′-monophosphate itself has a yellow tint. The adsorption and desorption of riboflavin phosphate (yellow colored) was examined on extracted human teeth. Specimens (N=8) showed an average b* value of 21.42 before a 20 minute treatment with an aqueous 0.01% riboflavin phosphate solution. The average b* value after treatment was 22.57 (delta b=1.15). The specimens were subsequently exposed to ambient light for 4 hours, after which an average b* of 21.43 (delta b=0.01 from original b*) was obtained. The results demonstrate the reversible adsorption of the yellow colored riboflavin phosphate to the enamel surface, which would explain any transient increase in yellowness following treatment with a riboflavin phosphate prototype.

As such, the initial bleaching results shown in Table 3 are a reflection of residual riboflavin 5′-monophosphate on the surface of the teeth, resulting in a transient discoloration effect. As the residual riboflavin 5′-monophosphate is depleted from the tooth surface, for example by dilution with saliva and/or abrasion, the discoloration effect disappears and the whiteness of the teeth can be seen.

### Example 4

### Impact of pH on Enhanced Generation of Reactive Species in Tooth Whitening

The following stock solution was prepared:
4A) Salicylate stock solution: 0.10 grams of sodium salicylate were added to a 100 milliliter volumetric flask and diluted to volume with purified water.

The following four formulations were then prepared at pH 5.0, 6.0, 6.5, 7.0, 7.5, 8.0, and 9.0 using the stock solution:
4B) Control solution: 5.0 milliliter of solution 4A was transferred to a 100 milliliter low-actinic volumetric flask and diluted to volume with 0.05 M phosphate buffer at the specified pH.
4C) 0.6% hydrogen peroxide solution: 5.0 milliliter of solution 4A and 20 milliliter of 3.0% hydrogen peroxide were added to a low-actinic 100 milliliter volumetric flask and then diluted to volume with 0.05 M phosphate buffer at the specified pH.
4D) 0.3 mM Riboflavin: 5.0 milliliter of solution 4A and 5.0 mg of riboflavin powder was added to a 100 milliliter low-actinic volumetric flask and diluted to mark with 0.05 M phosphate buffer at the specified pH.
4E) 0.3 mM Riboflavin plus 0.6% H2O2: 5.0 milliliter of solution 4A, 20.0 milliliter of 3.0% hydrogen peroxide and 5.0 mg of riboflavin powder was added to a 100 milliliter low-actinic volumetric flask and diluted to mark with 0.05 M phosphate buffer at the specified pH.

Solutions 4B to 4E at each pH were irradiated with visible light (450 nm +/−25 nm, 2 mW) for thirty minutes and the hydroxylation of salicylate with hydroxide radical was monitored through a loss of salicylate chromatographically using the conditions described in Example 1. The results are shown on Table 4.

TABLE 4
Hydroxylation of salicylate. (errors typically +/− 0.25 mg salicylate
oxidized)

| | milligram of salicylate | | |
|---|---|---|---|
| | oxidized relative to | | |
| | control (4B) | | |
| | | | 0.3 mM Riboflavin + 0.6% |
| | 0.6% peroxide | 0.3 mM Riboflavin | peroxide |
| pH | 4C | 4D | 4E |
| 5.0 | 0.03 | 2.08 | 1.50 |
| 6.0 | −0.09 | 3.02 | 2.97 |
| 6.5 | −0.04 | 3.96 | 3.43 |
| 7.0 | 0.00 | 4.24 | 3.97 |
| 7.5 | −0.11 | 3.29 | 3.80 |
| 8.0 | 0.05 | 2.59 | 4.01 |
| 9.0 | −0.04 | 1.02 | 3.80 |

The table shows that at all pH values, more reactive species are generated with irradiated (450 nm) riboflavin formulations (4D) or riboflavin plus peroxide formulations (4E) when compared to the peroxide control formulations (4C). The table also shows that the riboflavin formulations generate significantly more reactive species in the pH range of about 5.0 to about 6.5, while the riboflavin plus peroxide formulations generated comparable or significantly more reactive species in the pH range of about 7.0 to 9.0. Greater than 9 was not tested but it is assumed that synergy would be observed in this range, but applicability to oral treatments is diminished as the pH goes well above 9.0. The data indicate that the riboflavin-peroxide composition is ideally formulated at pH greater than 6.5 due to the enhancement of hydroxyl radical generation and whitening activity. The activity of the combination is synergistically enhanced compared to either riboflavin or peroxide alone.

### Example 5

### Impact of Light Power Enhanced Generation of Reactive Species in Tooth Whitening

The following three formulations were prepared:
5A) Control solution: 5.0 milliliter of formulation 4A (Example 4) was transferred to a 100 milliliter low-actinic volumetric flask and diluted to volume with 0.05 M phosphate buffer at pH 7.5.
5B) 0.6% hydrogen peroxide solution: 5.0 milliliter of solution A and 20 milliliter of 3.0% hydrogen peroxide were added to a low-actinic 100 milliliter volumetric flask and then diluted to volume with 0.05 M phosphate buffer at pH 7.5.
5C) 0.3 mM Riboflavin plus 0.6% H2O2: 5.0 milliliter of solution A, 20.0 milliliter of 3.0% hydrogen peroxide and 5.0 mg of riboflavin powder was added to a 100 milliliter low-actinic volumetric flask and diluted to mark with 0.05 M phosphate buffer at pH 7.5.

Solutions 5A to 5C were irradiated with visible light (450 nm +/−25 nm) at 2, 10, and 25 mW) for thirty minutes and the hydroxylation of salicylate with hydroxide radical was monitored through a loss of salicylate chromatographically using the conditions described in Example 1. The results are show on Table 5.

TABLE 5
Hydroxylation of salicylate.

| | | milligrams of | |
|---|---|---|---|
| | | salicylate oxidized | |
| | | relative to control | |
| | 2 | (5A) | 25 |
| | mW/cm2 | 10 mW/cm2 | mW/cm2 |
| 0.6% peroxide (5B) | 0.39 | 0.39 | 0.43 |
| 0.6% peroxide + Riboflavin | 2.76 | 4.26 | 4.97 |
| (5C) | | | |

The table shows that at all levels of light power, the riboflavin plus peroxide formulations (5C) generated more reactive species when compared to the peroxide control formulations (5B). The table also shows that although as the level of light power increased, the riboflavin plus peroxide formulations generated more reactive species, low light power levels are very effective in producing radicals. Thermal sample heating was not observed.

### Example 6

### Impact of Enhanced Generation of Reactive Species on Tooth Whitening

A prototype gel formulation containing riboflavin and peroxide was prepared and evaluated in-vitro in a human tooth whitening model. The components of the formulation are shown on Table 6a.

TABLE 6a
Prototype gel formulation.

| Ingredient | Total % |
|---|---|
| Water, purified | 91.2 |
| CARBOPOL 974P | 2.00 |
| Hydrogen Peroxide | 3.2 |
| Sodium phosphate, monobasic | 0.40 |
| Sodium phospate, dibasic | 0.80 |
| Sodium saccharin | 0.10 |
| Cremophor RH 40 | 2.00 |
| Mint Flavor | 0.30 |
| Riboflavin | 0.005 |
| Sodium Hydroxide | Add to pH 7.4 |

The prototype formulation listed above was applied to human molar enamel and exposed either to ambient light or to 450 nm irradiation at 7.5 mW/cm2 for 20 minutes per treatment. The color of the stain on the bovine teeth was measured by taking diffuse reflectance absorbance readings with a SpectroShade Micro spectrophotometer (MHT, Italy). Absorbance measurement over the entire visible color spectrum were obtained using the CIELAB color scale (Commission International de L'Eclairage, 1978 and 1986). This scale quantifies color according to 3 parameters, L* (lightness-darkness); a* (red-green); and b* (yellow-blue) with increasing L* and decreasing b* being more aesthetically pleasing and desirable. Table 6b and 6c show the change in the L* values (␣L) and b* values (␣b) with each treatment for the formulation with exposure to ambient light or 450 nm irradiation at 7.5 mW/cm2.

TABLE 6b
Human teeth bleaching.

| | □L Prototype A with | □L Prototype A with |
|---|---|---|
| Treatment # | 450 nm Light | Ambient Light |
| 1 | 1.04 | 0.92 |
| 2 | 1.20 | 0.79 |
| 3 | 1.40 | 1.19 |
| 4 | 1.71 | 1.13 |
| 5 | 1.88 | 1.41 |
| 6 | 2.06 | 1.63 |
| 7 | 1.81 | 1.24 |
| 8 | 2.18 | 1.52 |
| 9 | 2.23 | 1.70 |
| 10 | 1.94 | 1.69 |
| 11 | 2.35 | 1.75 |
| 12 | 2.35 | 1.75 |
| 13 | 2.32 | 1.87 |
| 14 | 2.45 | 1.97 |

TABLE 6c
Human teeth bleaching.

| | □b Prototype A with | □b Prototype A with |
|---|---|---|
| Treatment # | 450 nm Light | Ambient Light |
| 1 | −0.36 | 0.85 |
| 2 | −1.01 | 0.53 |
| 3 | −1.25 | 0.47 |
| 4 | −1.67 | 0.18 |
| 5 | −2.09 | −0.23 |
| 6 | −2.34 | −0.44 |
| 7 | −2.69 | −0.87 |
| 8 | −2.84 | −0.80 |
| 9 | −3.04 | −0.93 |
| 10 | −3.77 | −1.36 |
| 11 | −3.54 | −1.59 |
| 12 | −3.54 | −1.59 |
| 13 | −3.87 | −1.46 |
| 14 | −4.02 | −1.58 |

The table shows an enhancement in tooth whitening with each treatment, and superior enhancement in tooth whitening with the combination of riboflavin and peroxide at 450 nm light when compared to ambient light.

### Example 7

### Impact of Riboflavin-Containing Films (Strips) on Tooth Whitening

A thin film was cast from the following formulation:

| Ingredient | Added % |
|---|---|
| Ethanol | 64.89 |
| Peroxydone K-90 (ISP) | 31.40 |
| PEG-400 | 3.00 |
| PEG-4500 | 0.50 |
| L-Menthol Crystals | 0.10 |
| Sodium Saccharin | 0.10 |
| Riboflavin | 0.01 |

The formulation was prepared by adding PEG-400 and PEG-4500 slowly to ethanol at 40° C. during agitation from a speed mixer. After complete incorporation of the polymers, the mixture was cooled and peroxydone was added slowly. Next, menthol, saccharin and riboflavin were added and the bulk was mixed until homogeneous.

To make the dental strip, film was first cast onto a Teflon substrate to a wet thickness of 32 mils and allowed to dry for 30 minutes in a 60° C. oven.

One cm2 strips were cut from the film and was applied to human enamel specimens. The specimens were exposed either to ambient light or to 450 nm irradiation at 7.5 mW/cm2 for 20 minutes per treatment. The color of the stain on the bovine teeth was measured by taking diffuse reflectance absorbance readings with a SpectroShade Micro spectrophotometer (MHT, Italy). Absorbance measurement over the entire visible color spectrum were obtained using the CIELAB color scale (Commission International de L'Eclairage, 1978 and 1986). This scale quantifies color according to 3 parameters, L* (lightness-darkness); a* (red-green); and b* (yellow-blue) with increasing L* and decreasing b* being more aesthetically pleasing and desirable. Table 7 shows the change in the L* values (□L) and b* values (□b) with each treatment for the formulation with exposure to ambient light or 450 nm irradiation at 7.5 mW/cm2.

TABLE 7
Human teeth bleaching.

| | □L | □L | | |
|---|---|---|---|---|
| | Prototype | Prototype | | |
| | with | with | □b | □b |
| | 450 nm | Ambient | Prototype with | Prototype with |
| Treatment # | Light | Light | 450 nm Light | Ambient Light |
| 1 | −0.04 | −0.24 | −2.60 | −1.54 |
| 2 | 0.50 | 0.28 | −3.51 | −1.92 |
| 3 | 0.62 | 0.36 | −3.91 | −2.37 |
| 4 | 0.73 | 0.57 | −4.23 | −2.50 |
| 5 | 0.44 | 0.41 | −4.81 | −2.90 |
| 6 | 0.68 | 0.34 | −5.00 | −3.07 |
| 7 | 1.21 | 1.16 | −4.61 | −2.39 |
| 8 | 1.29 | 1.05 | −5.08 | −2.63 |
| 9 | 1.48 | 1.36 | −5.07 | −2.57 |
| 10 | 1.50 | 1.49 | −5.29 | −2.62 |
| 11 | 1.66 | 1.41 | −5.54 | −3.24 |
| 12 | 1.91 | 1.63 | −5.64 | −3.38 |
| 13 | 1.56 | 1.46 | −5.79 | −3.22 |
| 14 | 1.99 | 1.76 | −5.99 | −3.36 |

The table shows an enhancement in tooth whitening with each treatment, and superior enhancement in tooth whitening with the combination of riboflavin and peroxide at 450 nm light when compared to ambient light. These in-vitro results were subsequently validated in a clinical study (N=29).

### Example 8

### Comparison Example. Use of Methylene Blue and Toluidine Blue O with Red Light to Activate Peroxide

The following stock solution was prepared:
8A) Salicylate stock solution: 0.10 grams of sodium salicylate were added to a 100 milliliter volumetric flask and diluted to volume with purified water.

The following four formulations were prepared using the stock solution:
8B) Control solution: 5.0 milliliter of solution A was transferred to a 100 milliliter low-actinic volumetric flask and diluted to volume with 0.05 M phosphate buffer at pH 8.0.
8C) 0.6% hydrogen peroxide solution: 5.0 milliliter of solution A and 20 milliliter of 3.0% hydrogen peroxide were added to a low-actinic 100 milliliter volumetric flask and then diluted to volume with 0.05 M phosphate buffer at pH 8.0.
8D) 0.6% hydrogen peroxide plus methylene blue: 5.0 milliliter of solution A, 20.0 milliliter of 3.0% hydrogen peroxide and 5.0 mg of methylene blue was added to a 100 milliliter low-actinic volumetric flask and diluted to mark with 0.05 M phosphate buffer at pH 8.0.
8E) 0.6% hydrogen peroxide+toluidine blue O: 5.0 milliliter of solution A, 20.0 milliliter of 3.0% hydrogen peroxide and 5.0 mg of toluidine blue O was added to a 100 milliliter low-actinic volumetric flask and diluted to mark with 0.05 M phosphate buffer at pH 8.0.

Formulations 8B to 8E were irradiated with visible light (632 nm, dispersed He—Ne laser light, 10 mW/cm2) for thirty minutes and the hydroxylation of salicylate with hydroxide radical was monitored through a loss of salicylate chromatographically using the conditions described in Example 1. The results are shown on Table 8.

TABLE 8
Hydroxylation of Salicylate

Salicylate oxidized (mg)

| | |
|---|---|
| Control (8B) | 0.00 |
| 0.6% peroxide (8C) | 0.03 |
| Methylene blue + 0.6% peroxide (8D) | 0.05 |
| Toluidine Blue 0 + 0.6% peroxide (8E) | 0.04 |

These results demonstrate that both methylene blue and toluidine blue O with the combination of peroxide are inferior to riboflavin and peroxide in producing reactive bleaching species when irradiated with their optimal absorbance frequencies.

### Example 9

### Use of Rose Bengal with Green Light to Activate Peroxide

The following stock solution was prepared:
9A) Salicylate stock solution: 0.10 grams of sodium salicylate were added to a 100 milliliter volumetric flask and diluted to volume with purified water.

The following four formulations were prepared at pH 5.0, 6.0, 7.0, 8.0, and 9.0 using stock solution 9A:
9B) Control solution: 5.0 milliliter of solution 9A was transferred to a 100 milliliter low-actinic volumetric flask and diluted to volume with 0.05 M phosphate buffer at the specified pH.
9C) 0.6% hydrogen peroxide solution: 5.0 milliliter of solution 9A and 20 milliliter of 3.0% hydrogen peroxide were added to a low-actinic 100 milliliter volumetric flask and then diluted to volume with 0.05 M phosphate buffer at the specified pH.
9D) Rose Bengal solution: 5.0 milliliter of solution 9A and 6.6 mg of rose Bengal was added to a 100 milliliter low-actinic volumetric flask and diluted to mark with 0.05 M phosphate buffer at the specified pH.
9E) 0.6% hydrogen peroxide+rose bengal: 5.0 milliliter of solution 9A, 20.0 milliliter of 3.0% hydrogen peroxide and 6.6 mg of rose bengal was added to a 100 milliliter low-actinic volumetric flask and diluted to mark with 0.05 M phosphate buffer at the specified pH.

Formulations 9B to 9E at each pH were irradiated with visible light (550 +/−25 nm, green LED, 2 mW/cm2) for thirty minutes and the hydroxylation of salicylate with hydroxide radical was monitored through a loss of salicylate chromatographically using the conditions described in Example 1. The results are shown on Table 9.

TABLE 9
Hydroxylation of Salicylate

| | milligram of salicylate | | |
|---|---|---|---|
| | oxidized relative to control | | |
| | 0.6% | | 0.3 mM rose bengal + 0.6% |
| pH | peroxide N | 0.3 mM rose bengal O | peroxide P |
| 5 | 0.0 | 0.1 | 0.1 |
| 6 | 0.0 | 0.1 | 0.2 |
| 7 | 0.0 | 0.1 | 0.2 |
| 8 | 0.0 | 0.2 | 0.3 |
| 9 | 0.1 | 0.2 | 0.2 |

These results demonstrate a minor enhancement when irradiated with their optimal absorbance frequency in radical generation upon the combination of rose Bengal with peroxide, although the effect is inferior to that of the riboflavin-peroxide combination.

### Example 10

### Tooth Whitening with Low/No Peroxide Formulations

Three prototype gel formulations were prepared and evaluated in-vitro in a human tooth whitening model. The components of the formulations are shown on Table 10a:

TABLE 10a
Components in prototype gel formulations.

| Ingredient | % in A | % in B | % in C |
|---|---|---|---|
| Water, purified | 94.3 | 94.4 | 94.3 |
| CARBOPOL 974P | 2.0 | 2.0 | 2.0 |
| Hydrogen Peroxide | 0.1 | 0.0 | 0.1 |
| Sodium phosphate, monobasic | 0.4 | 0.4 | 0.4 |
| Sodium phospate, dibasic | 0.8 | 0.8 | 0.8 |
| Sodium saccharin | 0.1 | 0.1 | 0.1 |
| Cremophor RH 40 | 2.0 | 2.0 | 2.0 |
| Mint Flavor | 0.3 | 0.3 | 0.3 |
| Riboflavin | 0 | 0.005 | 0.005 |
| Sodium Hydroxide | Add to pH 7.4 | Add to pH | Add |
| | | 7.4 | to pH 7.4 |

The prototype formulations listed above were applied to human molar enamel and exposed to 450 nm irradiation at 7.5 mW/cm² for 20 minutes per treatment and L*a*b* values were measured after each treatment. Table 10b and 10c show the change in the L* values (ΔL) and b* values (Δb) with each treatment for the formulation with exposure to ambient light or 450 nm irradiation at 7.5 mW/cm².

TABLE 10b
Human teeth bleaching.

| Treatment | □L - A | □L - B | □L - C |
|---|---|---|---|
| 1 | 0.51 | 0.46 | 0.51 |
| 2 | 0.82 | 0.37 | 0.70 |
| 3 | 0.56 | 0.37 | 0.59 |
| 4 | 0.60 | 0.51 | 0.66 |
| 5 | 0.60 | 0.76 | 1.05 |
| 6 | 0.81 | 0.64 | 0.96 |
| 7 | 0.74 | 0.64 | 0.92 |
| 8 | 0.74 | 0.52 | 0.91 |
| 9 | 0.89 | 0.62 | 0.94 |
| 10 | 0.81 | 0.59 | 0.80 |
| 11 | 0.79 | 0.67 | 1.09 |
| 12 | 0.60 | 0.54 | 0.98 |
| 13 | 0.83 | 0.72 | 1.21 |

TABLE 10c
Human teeth bleaching.

| Treatment | □b - A | □b - B | □b - C |
|---|---|---|---|
| 1 | −0.36 | −0.20 | −0.58 |
| 2 | −0.45 | −0.18 | −0.91 |
| 3 | −0.55 | −0.29 | −1.09 |
| 4 | −0.56 | −0.20 | −1.11 |
| 5 | −0.59 | −0.13 | −1.54 |
| 6 | −0.82 | −0.43 | −1.45 |
| 7 | −0.84 | −0.42 | −1.62 |
| 8 | −0.97 | −0.52 | −1.75 |
| 9 | −0.91 | −0.40 | −1.76 |
| 10 | −1.08 | −0.56 | −1.95 |
| 11 | −1.09 | −0.49 | −1.89 |
| 12 | −1.17 | −0.58 | −2.08 |
| 13 | −1.17 | −0.58 | −2.08 |

Results from these experiments demonstrated an enhancement in tooth whitening with the combination of the riboflavin and low level peroxide when compared to either the riboflavin or peroxide control.

### Example 11

### Anti-Microbial Enhancement with Riboflavin-Peroxide Formulations

Three formulations were prepared. The components of the formulations are shown on Table 11.

TABLE 11
Components of anti-microbial formulations

| | 11A | 11B | 11C |
|---|---|---|---|
| Water | 99.5 | 99.5 | 99.6 |
| Riboflavin | 0.005 | 0 | 0.005 |
| Hydrogen Peroxide | 0.1 | 0.1 | 0 |
| Sodium Phosphate, | 0.4 | 0.4 | 0.4 |
| monobasic | | | |
| Sodium Hydroxide | adjust to pH 7.0 | adjust to pH 7.0 | adjust |
| | | | to pH 7.0 |

In vitro assessment of Microbial Kill Time (MKT) with planktonic oral pathogen Streptococcus mutans ATCC 35668, an organism associated with caries, demonstrated a 3.59-log reduction in total viable cell counts after an exposure time of 5 minutes to composition 11A. As exposure to light was increased to 10 minutes and 30 minutes, the log reduction increased to >4.95 at both of these time points. Exposure to composition 11B in the presence of light resulted in a 1.81-log reduction in total viable cell counts after 30 minutes. No antimicrobial effect was observed with composition 11C.

MKT data suggest that 0.1% hydrogen peroxide solution alone in the presence of light has antimicrobial activity but this activity was significantly enhanced when the solution was combined with 0.005% riboflavin.

### Example 12

### Clinical Tooth Whitening with Riboflavin-Peroxide Compositions

A prototype gel formulation was prepared and evaluated clinically for tooth whitening. The components of the formulation are shown on Table 12a:

TABLE 12a
Components in prototype gel formulation.

| Ingredient | Total % |
|---|---|
| Water, purified | 90.4 |
| CARBOPOL 974P | 2.00 |
| Hydrogen Peroxide | 4.0 |
| Sodium phosphate, monobasic | 0.40 |
| Sodium phospate, dibasic | 0.80 |
| Sodium saccharin | 0.10 |
| Cremophor RH 40 | 2.00 |
| Mint Flavor | 0.30 |
| Riboflavin | 0.005 |
| Sodium Hydroxide | Add to pH 7.4 |

Each subject's maxillary arch was treated with the prototype formulation listed above, applied in a custom whitening tray, for 20 minutes per day for a total of 14 treatments. The subjects from group A (N=15) remained in a dark room during treatment while the maxilliary arches of the subjects from group B (N=14) were exposed to 450 nm irradiation at 10.0 mW/cm². L*a*b* values were measured at baseline, 24 hours after treatments 3, 5, 7, 10, and 14, and 7 days post-treatment. Table 12b and 12c show the change in the L* values (ΔL) and b* values (Δb) with each treatment for groups A and B.

TABLE 12b
Clinical tooth whitening results - □L

| | Delta L - | Delta L - |
|---|---|---|
| Treatment # | Group A | Group B |
| 3 | 0.29 | 0.67 |
| 5 | 0.67 | 0.99 |
| 7 | 0.61 | 0.86 |
| 10 | 0.78 | 1.06 |
| 14 | 0.80 | 1.15 |
| 7 days post-treatment | 0.98 | 1.29 |

TABLE 12c
Clinical tooth whitening results - □b

| | Delta b - | Delta b - |
|---|---|---|
| Treatment # | Group A | Group B |
| 3* | −0.49 | −0.93 |
| 5* | −0.56 | −1.18 |
| 7* | −0.74 | −1.64 |
| 10* | −0.81 | −2.15 |
| 14* | −1.05 | −2.57 |
| 7 days post-treatment* | −0.94 | −2.38 |

*= statistically significant difference between groups

Results from this clinical study demonstrate an enhancement in tooth whitening with the combination of the riboflavin, peroxide, and blue light when compared to riboflavin and peroxide without light. Of note is that □L and □b do not change from treatment 14 to 7 days post-treatment, demonstrating that none of the increase in whiteness is a result of dehydration of enamel, as is usually the case with light-enhanced whitening treatments due to the high-power lamps that are typically used.

We claim:

1. A tooth whitening composition capable of being photoactivated, comprising:
a safe and effective amount of riboflavin or a derivative of riboflavin,
from about 0.01 percent to about 50 percent by weight of a bleaching agent; and
a water-soluble liquid phase.

2. The composition of claim 1 comprising from about 0.0001% to about 0.5% by weight of the riboflavin or derivative of riboflavin.

3. The composition of claim 2 wherein the bleaching agent is selected from the group consisting of peroxides, metal chlorites, perborates, percarbonates, peroxyacids, persulfates, peroxyacids and organic peroxides.

4. The composition of claim 2 wherein the bleaching agent is selected from the group consisting of hydrogen peroxide, urea peroxide, calcium peroxide, carbamide peroxide, polyvinylpyrolidone hydrogen peroxide complexes and copolymers of polyvinylpyrolidone complexed with hydrogen peroxide.

5. The composition of claim 4 comprising from about 0.001 percent to about 0.5 percent by weight of the riboflavin or derivative of riboflavin.

6. The composition of claim 5 comprising from about 0.1 percent to about 15 percent by weight of hydrogen peroxide.

7. The composition of claim 2 having a pH of greater than about 6.5.

8. The composition of claim 3 wherein the water-soluble liquid phase is selected from the group consisting of water, ethanol, propanol, polyalkylene glycols, glycerin, sorbitol, xylitol, butylene glycol, polyethylene glycol and propylene glycol.

9. The composition of claim 2 further comprising from about 0.1 percent to about 10 percent by weight of a thickening agent.

10. The composition of claim 2 further comprising a water-insoluble solid phase.

11. A method of whitening teeth, comprising:
applying to the surface of teeth a photoactivatable tooth whitening composition comprising a safe and effective amount of riboflavin or a derivative of riboflavin, from about 0.1 to about 50 percent by weight of a bleaching agent and a water-soluble liquid phase,
exposing the tooth whitening composition applied to the teeth to light energy under conditions effective to photoactivate the tooth whitening composition; and
maintaining the photoactivated tooth whitening composition in contact with the teeth under conditions effective to whiten the teeth.

12. The method of claim 11 wherein the wavelength of the light is between about 250 and about 700 nm.

13. The method of claim 11 wherein the power of the light is between about 0.1 mW/cm2 and about 100 mW/cm2.

14. The method of claim 12 wherein the tooth whitening composition comprises from about 0.0001% to about 0.5% by weight of the riboflavin or derivative of riboflavin.

15. The method of claim 11 wherein the tooth whitening composition is applied to the teeth more than once.

16. The method of claim 11 wherein the tooth whitening composition is maintained in contact with the teeth for between about 30 seconds to about 12 hours.

17. The method of claim 16 wherein the tooth whitening composition is applied to the teeth more than once.

18. The method of claim 11 wherein the duration of exposure of the tooth whitening composition to the light is from about 30 seconds to about 60 minutes.

19. The method of claim 18 wherein the tooth whitening composition is applied to the teeth more than once.

20. A tooth whitening delivery system, comprising:
an integral carrier; and
a photoactivable tooth whitening composition applied to the integral carrier, the photoactivable tooth whitening composition comprising a safe and effective amount of riboflavin or a derivative of riboflavin, from about 0.1 to about 50 percent by weight of a bleaching agent and a water-soluble liquid phase.

21. The delivery system of claim 20 comprising from about 0.0001% to about 0.5% by weight of the riboflavin or derivative of riboflavin.

22. The delivery system of claim 21 wherein the bleaching agent is selected from the group consisting of peroxides, metal chlorites, perborates, percarbonates, peroxyacids, persulfates, peroxyacids and organic peroxides.

23. The delivery system of claim 20 wherein the integral carrier is selected from the group consisting of a strip of material, a dental tray and a sponge material.

24. The delivery system of claim 21 wherein the integral carrier is a strip of material.

25. The delivery system of claim 21 wherein the integral carrier is a dental tray.
